(19) United States
(12) Patent Application Publication
(10) Pub. No.: US 2005/0008583 A1
(43) Pub. Date: Jan. 13, 2005

(54) Material and system for gingival retraction and other applications

(76) Inventor: Sander White, Broomall, PA (US)

Correspondence Address: Joan T. Kluger;IP Department, Schnader Harrison Segal & Lewis LLP, 1600 Market Street, Suite 3600, Philadelphia, PA, 19103, US

(21) Appl. No.: 10/885,851

(22) Filed: Jul. 7, 2004

Related U.S. Application Data
(60) Provisional application No. 60/486,599, filed on Jul. 11, 2003.
(60) Provisional application No. 60/501,588, filed on Sep. 8, 2003.

(51) Int. Cl. A61K 7/16

(52) U.S. Cl. 424/49

(57) ABSTRACT

A gingival retraction material and method of fabrication. The material comprises a carrying medium, retraction medium, and an anti-evaporation component. Preferably the carrying and retraction media are a clay such as kaolin, and the anti-evaporation component is glycerin. Additional components may be added to the material such as aluminum chloride, aluminum sulfate, ferric chloride, ferric subsulfate, styptics, haemostatic agents, coagulants coloring and flavoring. The material may be packaged in a kit with components to apply the material. Further disclosed is the method for making the material. Also disclosed is a delivery system for medication that includes a carrying medium, anti-evaporation component and a medication.

This application is based, and claims priority to, provisional application having Ser. No. 60/486,599, filed on of Jul. 11, 2003, and provisional application having Ser. No. 60/501,588, filed on Sep. 8, 2003, both entitled Material and System for Gingival Retraction and Other Applications.

### BACKGROUND OF THE INVENTION

Gingival retraction is the process of moving the gingivae away from the tooth surface. Displacement of the marginal gingivae is often required for dental procedures. Such displacement may cause undesirable bleeding or oozing.

Gingival retraction may be accomplished by inserting material, such as cotton cord, between the tooth and gingivae. The material may be impregnated with substances to promote retraction and reduce bleeding. Impregnated, spongy materials fashioned to encircle the tooth have also been used. These methods are painful and often cause significant bleeding.

Liquid or pliable materials, such as pastes, have also been used to retract the gingivae. These materials may be inserted through a needle. The materials often harden very quickly, increasing the difficulty of insertion.

Accordingly, there is a need for a gingival retraction material that is easily and accurately inserted, and which does not harden prematurely.

### SUMMARY OF THE INVENTION

A gingival retraction material is disclosed comprising a carrying medium, retraction medium, and an anti-evaporation component. Preferably the carrying and retraction media are a clay such as kaolin, and the anti-evaporation component is glycerin. Additional components may be added to the material such as styptics (for example, aluminum chloride, aluminum sulfate, ferric chloride and ferric subsulfate), haemostatic agents, coagulants, coloring and flavoring.

Also disclosed is a kit that includes the packaged material and components to apply the material. Further disclosed is the method for making the material.

The invention also includes a delivery system for other medication that includes a carrying medium, anti-evaporation component and the medication, such as an antibiotic.

### DETAILED DESCRIPTION OF THE INVENTION

A gingival retraction material is disclosed that comprises, in a preferred embodiment, kaolin, aluminum chloride, water, and glycerin. Preferably the kaolin or clay is micronized. Flavoring and/or coloring may also be added. Materials comparable to the above-listed components may be substituted. The inventive material in a broad embodiment comprises: a carrying medium, retraction medium (which may be the same as the carrying medium), and an anti-evaporation component. The material may also comprise one or more of the following: styptics, haemostatic agents, coagulants coloring and flavoring

The material may be dispensed with or without a dispensing device such as a gun. The preferred dispensing method is to place the medium on a cotton roll, or similar applicator, and to press the material over the object tooth. The material can also be tamped in to place with a small piece of cotton or other appropriately sized and textured instrument.

In an illustrative embodiment, the material formula is as follows:
1. Kaolin powder 80% by weight
2. Aluminum Chloride 15% by weight.
3. Water/Glycerin sufficient to produce a heavy plastic consistency
4. Flavoring/color as desired.

A general illustrative composition range for the retraction/carrying medium is from about 70% to about 90%, and for aluminum chloride, about 10% to about 20%.

After mixing these ingredients to a heavy paste, the mixture is heated (preferably in a microwave oven) to allow for better solubility of the ingredients. Additional powder mixture (i.e. ingredients 1&2 in proportion) is added to the maximum degree possible to produce the heaviest consistency.

Any powder that will deliver the consistency and that will be miscible with the other ingredients might be substituted for kaolin. A gum base or flour and salt mixture may also be used as a base.

The choice of glycerin as a liquid medium is based on its ability to provide a flowable paste, its ability to impart a somewhat sweet taste and its resistance to evaporation, which allows the product a good shelf life and eliminates the drying out of the material if it is exposed to the air. Other liquids may also fulfill some or all of these qualities and might therefore be used. An illustrative example is propylene glycol.

All of the materials used should be of medical grade quality and where appropriate of USP grade when used for gingival retraction and/or as an astringent for example. Other applications of the material are possible, such as a means of stopping bleeding after minor gingival surgical procedures such as gingivectomy and after removing temporary restorations prior to luting of permanent restorations , or delivery of other medications into confined spaces or space that can be confined. Examples of medications that may be deliverable via the material include, but are not limited to, antibiotics and medications used in treatment of abrasion wounds. The applications mentioned are not limited to dental applications, but may be used in medical or vetinarial application.

The viscosity of this material may be adjusted, to a desired consistency by addition of water and/or glycerin.

The invention includes a packaged product. The preferred packaging method is a container, such as a simple small plastic “tub” that would hold approximately one gram—sufficient to treat 2-3 teeth. It would be sealed so as to provide an individual “dose pack” for control of contamination issues. The manufactured product would be produced to the standards necessary to satisfy any sterility and toxicity concerns. Of course there is a concern of cross contamination that would be easily and cheaply solved by the dose packaging mentioned. Other packaging methods are within the scope of the invention, such as larger containers, syringe delivery or carpule (cartridge) delivery.

The inventive formulation is advantageous for numerous reasons. Most notably, the formulation of water and glycerin prevents or inhibits evaporation and drying out of product. This improves storage life and prevents waste.

The liquid formulation further allows the material to flow easily into sulcus or wound and then be absorbed by such materials as a cotton roll to increase viscosity and thereby be a more effective retraction medium.

Kaolin and other comparable bases which act as the carrying medium and retraction medium are very versatile and easy to manufacture with a variety of liquid media. Flavoring agents may easily be added to make the material more palatable. Different coloring agents may be incorporated to easily identify different formulations. Different styptics, haemostatic agents and coagulants may be incorporated in the base to provide for doctors' preferences. These could include, but are not be limited to, aluminum chloride, aluminum sulfate, ferric chloride, ferric subsulfate, and all other such materials.

The ability to use the material with a cotton roll, such as Comprecap™ or other similar product, is very beneficial. Application is faster, easier and more effective than injection methods as it provides a means to compress material into the sulcus or wound. Because the material can be provided at a much cheaper cost the “apparent” waste is of little consequence.

The material may be packaged in a “kit” including cotton roll slices or other application devices. A kit could include other related components such as impression devices. The material may also be pre-applied to the application device for even greater convenience, or otherwise incorporated into a delivery device.

Different viscosities can be produced as a method of customizing to doctors' preferences—also as a means of segmenting the market.

Packaging can be in various forms such as in a small tub similar to Prophy paste or a flexible tube like toothpaste, or in “single dose” tips like composite, or to be loaded in a CR syringe tip. It can be provided in various forms, again to appeal to doctors' preferences and to segment the market.

The inventive product can also be used to control bleeding, such as after gingivectomy prior to cosmetic procedures.

While the invention has been described by illustrative embodiments, additional advantages and modifications will occur to those skilled in the art. Therefore, the invention in its broader aspects is not limited to specific details shown and described herein. Modifications, for example, to the ratios of material components, uses of the material and packaging, may be made without departing from the spirit and scope of the invention. Accordingly, it is intended that the invention not be limited to the specific illustrative embodiments, but be interpreted within the full spirit and scope of the appended claims and their equivalents.

1. A gingival retraction material comprising:
a carrying medium;
retraction medium; and
an anti-evaporation component.

2. The gingival retraction material of claim 1 further comprising one or more components selected from the group consisting of aluminum chloride, aluminum sulfate, ferric chloride and ferric subsulfate.

3. The gingival retraction material of claim 1 further comprising one or more components selected from the group consisting of styptics, haemostatic agents, coagulants coloring and flavoring.

4. The gingival retraction material of claim 1 further comprising water.

5. The gingival retraction material of claim 1 wherein the retraction and carrying media together comprise between about 70% and 90% by weight of the material.

6. The gingival retraction material of claim 2 comprising aluminum chloride in the range of 70% and 90% by weight of the material.

7. The gingival retraction material of claim 1 wherein the anti-evaporation component is glycerin.

8. The gingival retraction material of claim 1 wherein the carrying medium is a gum base.

9. The gingival retraction material of claim 1 wherein the carrying medium is a mixture of water, flour and salt.

10. The gingival retraction material of claim 1 wherein the carrying and retraction material is clay.

11. The gingival retraction material of claim 10 wherein the clay is kaolin.

12. The gingival retraction material of claim 1 further comprising a container enclosing the material.

13. The gingival retraction material of claim 12 wherein the container encloses a quantity of material suitable for a single use.

14. A method of making a gingival retraction material comprising:
mixing a carrying medium, a retraction medium; and an anti-evaporation component to a heavy paste; and
heating the mixture.

15. The method of claim 14 further comprising adding additional carrying medium and/or retraction medium to adjust consistency.

16. The method of claim 14 further comprising packaging the material in a sealed container.

17. The method of claim 16 further comprising packaging the material in a quantity suitable for a single use.

18. The method of claim 14 further comprising incorporating the material into a delivery device.

19. A gingival retraction kit comprising:
a material having a carrying medium, a retraction medium; and an anti-evaporation component; and
one or more components for delivering the material.

20. The gingival retraction kit of claim 19 further comprising a package enclosing a quantity of the material sufficient for a single use.

21. The gingival retraction kit of claim 19 further comprising one or more impression devices.

22. A medication-delivering material comprising:
a carrying medium;
an anti-evaporation component; and
a medication

23. The medication-delivering material of claim 22 wherein the medication is an antibiotic.

24. The medication-delivering material of claim 22 further comprising one or more components selected from the group consisting of aluminum chloride, aluminum sulfate, ferric chloride and ferric subsulfate.

25. The medication-delivering material of claim 22 further comprising one or more components selected from the group consisting of coloring and flavoring.

26. The medication-delivering material of claim 22 wherein the carrying medium is kaolin and the anti-evaporation component is glycerin.

27. The medication-delivering material of claim 22 further comprising water.

28. The medication delivering-material of claim 22 wherein the medication is acoagulant.
